(19) United States
(12) Patent Application Publication
(10) Pub. No.: US 2009/0263368 A1
(43) Pub. Date: Oct. 22, 2009

(54) Genetic variations associated with psychiatric disorders

(75) Inventors: Thomas Bourgeron, Paris (FR); Jonas Melke, Goteborg (SE); Hany Goubran Botros, Le Pre Saint Gervais (FR); Jean-Marie Launay, Argenteuil (FR); Marion Leboyer, Paris (FR); Christopher Gillberg, Goteborg (SE); Pauline Chaste, Paris (FR); Richard Delorme, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: Institut Pasteur, Paris (FR)

(21) Appl. No.: 12/090,177

(22) PCT Filed: Oct. 13, 2006
(86) PCT No.: PCT/IB06/03935

§ 371 Date: Oct. 27, 2008

(30) Foreign Application Priority Data
Oct. 14, 2005 (CA) 2523399

(51) Int. Cl. A61K 38/45; C12Q 1/68; C07H 21/04; A61P 25/00; C07K 2/00

(52) U.S. Cl. 424/94.5; 435/6; 536/23.1; 536/24.1; 530/300

(57) ABSTRACT

The present invention relates to a method for determining likelihood of an individual of developing a psychiatric disorder. More particularly, the method of the invention is based on the identification of genetic variations in genes involved in the modulation of melatonin and their consequences on such pathway for the determination of whether an individual is susceptible of being afflicted by a psychiatric disorder such as autism spectrum disorders (ASD), Deficits and Hyper Activity Disorder (ADHD) and anorexia.

### FIELD OF THE INVENTION

The present invention relates to methods for determining likelihood of an individual of developing a psychiatric disorder. More particularly, the method of the invention is based on the identification of genetic variations in genes involved in the modulation of melatonin and their consequences on such pathway for the determination of whether an individual is susceptible of being afflicted by a psychiatric disorder such as autism, Deficits and Hyper Activity Disorder (ADHD) and anorexia and for the treatment of such a psychiatric disorder.

### BRIEF DESCRIPTION OF THE PRIOR ART

### Autism Spectrum Disorders (ASD) and Attention-Deficit/Hyperactivity Disorder (ADHD)

ASD and ADHD are among the most predominant psychiatric syndromes in children and seem to be strongly influenced by genes ¹⁻⁴. ASD is characterised by impairments in communication skills, social interaction and restricted, repetitive and stereotyped patterns of behaviour ⁵. ASD includes autistic disorder (classical autism), disintegrative disorder, pervasive development disorder not otherwise specified (PDD-NOS) and Asperger syndrome. The recurrence risk of autism in sib-ships is approximately 45 times greater than in the general population and twin studies have documented a higher concordance rate in monozygotic (60%-91%) than in dizygotic twins (0%-6%)¹. Furthermore, approximately 10% of individuals with autism have chromosomal rearrangements (e.g. duplication of 15q) or mutations in genes associated with other syndromes such as FMR1 (Fragile X syndrome), TSC1 and TSC2 (tuberous sclerosis) or NF1 (Neurofibromatosis).

ADHD is the most commonly diagnosed behavioural disorder in childhood affecting a relatively stable rate of 8-12% of all young children and likely represents an extreme of normal behaviour ⁶. It is a condition characterized by behavioural symptoms of inattention and/or hyperactivity-impulsivity, with onset in childhood ⁵. Such symptoms include restlessness, difficulty with organizing tasks, distractibility, forgetfulness, difficulty awaiting turns, and frequent interrupting. ADHD significantly impacts learning in school-age children and leads to impaired functioning throughout the life span. Family, twin, and adoption studies provided compelling evidence that genes play a strong role in mediating susceptibility to ADHD ^{3,4}.

Despite the clear clinical boundaries between these two disorders, there are behavioural, cognitive, and neurobiological deficits that suggest some degree of phenotypic overlap such as maladaptive social functioning and executive function deficits^{7,8}. Furthermore, there is a significant subgroup of children with ASD and symptoms of ADHD who respond to stimulant medication⁹, suggesting that common neurobiological mediators may be present in a subset of cases. Shared susceptibility genes for ASD and ADHD were also suggested from the results of genetic studies, which pointed at the same chromosomal regions using linkage analyses (e.g. 16p13 and 17p11)¹⁰ or chromosomal rearrangements (e.g. Xp22.3)^{11,12 13}.

### ASMT (Acetyl Serotonin Methyl Transferase) Gene and AA NAT (Arylalkylamine Acetyltransferase) Gene.

ASMT gene is located on the pseudo autosomal region 1 (PAR1), common to the X and the Y chromosome ¹⁴ ASMT encodes the enzyme HIOMT (Hydroxyindole-O-methyltransferase; EC 2.1.1.4), which catalyses the last step of melatonin biosynthesis. Melatonin (N-acetyl-5-methoxytryptamine) is the major secretory product of the pineal gland. It is produced through the conversion of tryptophan, first to serotonin, then N-acetylserotonin, and finally to melatonin^{15 16}. The synthesis of melatonin exhibits a pronounced circadian rhythm: its concentration in the body is typically lower during the day and reaches a maximal level at night in the darkness¹⁵. The regulation of melatonin production in the pineal gland involves especially norepinephrine (NE) couples to beta-adrenergic receptors¹⁷. Melatonin has been functionally linked to the regulation of circadian and seasonal rhythms, immune function, and is a powerful free radical scavenger and antioxidant ^{15,18}. Nevertheless, the consequence of the clock and calendar information that the melatonin cycle imparts to the organism is still not fully understood.

The enzyme AA.NAT is located before ASMT in the biosynthesis pathway and transforms the serotonin into N. acetyl serotonin.

A typical circadian rhythms are frequently observed in individuals with neuropsychiatric disorders and numerous studies have reported abnormal synthesis of melatonin or benefit from treatment with melatonin ^{19-21 22-25 26}. WO 02/076452 and WO 2004/028532 describe the use of melatonin in the treatment of ADHD. However, neither of these studies could discriminate between a cause or a consequence of the disorders.

### The Melatonin Receptors

In humans, two distinct melatonin receptors MTNR1A and MTNR1B have been reported so far. Both types of receptors were identified in a wide variety of tissues with different expression profiles. A third melatonin related receptor GPR50 was very recently identified. Although this receptor is structurally related to the melatonin receptors, with a 45% homology at the amino acid levels, it does not bind to melatonin. Nevertheless, the heterodimer MTNR1A/GPR50 abolishes high-affinity agonist binding and G protein coupling to the MTNR1A.

Activation of the MTNR1A leads to inhibition of forskolin stimulated cAMP formation, PKA activity, and phosphorylation of the cAMP-responsive element binding protein, a transcription factor. Activation of the MTNR1A also increases phosphorylation of mitogen-activated protein kinase and MEK1-2 and ERK1/2 probably leading to induction of synthesis of filamentous structures in non-neuronal tissues. Melatonin induction of filamentous structures in non-neuronal cells is dependent on expression of the human MTNR1A melatonin receptor. MTNR1A is coupled to parallel signal transduction pathways and regulates functional responses of melatonin in ion channels. Similar to the second messenger pathways of the MTNR1A receptor, activation of the MTNR1B also inhibits forskolin stimulated cAMP formation. Additionally coupling to this receptor can also lead to inhibition of cGMP formation.

Deletion of the large C-terminal tail of GPR50 suppresses the inhibitory effect of GPR50 on MT1 without affecting heterodimerization, indicating that this domain regulates the interaction of regulatory proteins to MT1. This effect appears to be specific for the GPR50/MT1 heterodimer since it was not observed for heterodimers with the closely related MT2. In mammals this receptor has been detected in various brain structures and peripheral tissues.

In humans, MTNR1A and MTNR1B were found in the hippocampus, throughout the cerebellar cortex in distinct cell populations. MTNR1A is localized in various areas related to dopaminergic behaviors, including Brodmann area 10 (i.e. prefrontal cortex), putamen, substantia nigra, amydala, and hippocampus. Melatonin may exert inhibitory effects, especially in the night, where melatonin levels are high.

There is thus a need for new tools marker associated with susceptibility to psychiatric disorders. Consequently, on the bases of both genetics and biochemical data, the inventors of the present invention propose that mutations in the ASMT gene cause an absence or a decrease of melatonin and confer an increased risk to neuropsychiatric disorders such as ASD and ADHD. The inventors thus demonstrate that some genetic variations in genes involved in melatonin biosynthesis cause an absence or a decrease of melatonin during childhood and adulthood and confer a risk to psychiatric disorders such as autism, ADHD and anorexia and thus provide new diagnostic and treatment methods with regards to such psychiatric disorders.

### SUMMARY OF THE INVENTION

One aspect of the invention is to provide a method for determining likelihood of an individual of developing a psychiatric disorder, comprising:
identifying at least one genetic variation in a gene involved in the modulation of melatonin and/or
assaying the mRNA transcript level of said gene and/or,
assaying the enzymatic activity of an enzyme involved in the melatonin biosynthesis pathway in a sample of said individual, and/or
assaying the melatonin level,
   whereby identification of a genetic variation and/or decrease in the enzymatic activity and/or a decrease in the transcript level of said gene and/or a decrease in melatonin level as compared to a psychiatric-free individual, is indicative of a likelihood that said individual develops a psychiatric disorder.

Another aspect of the invention concerns a composition for treating and/or preventing a psychiatric disorder in an individual having a defective gene involved in the modulation of melatonin, comprising an acceptable carrier and a therapeutically effective amount of a molecule promoting melatonin biosynthesis.

A further aspect of the invention is to provide a method for treating and/or preventing a psychiatric disorder in an individual having a defective gene involved in the modulation of melatonin comprising the administration in said individual of a composition of the invention.

The invention also provides the use of a composition comprising an acceptable carrier and a therapeutically effective amount of a molecule promoting melatonin biosynthesis, for the preparation of a medicament for treating and/or preventing a psychiatric disorder in an individual having a defective gene involved in the modulation of melatonin.

Yet, another aspect of the invention is to provide a method for treating and/or preventing ASD in an individual comprising the administration in said individual of a composition comprising melatonin or a functional derivative thereof.

The invention also provides the use of a composition comprising melatonin or a functional derivative thereof for the preparation of a medicament for treating and/or preventing ASD in an individual.

Yet a further aspect of the invention is to provide an isolated polynucleotide encoding a polypeptide or functional derivative thereof, characterized in that said polypeptide or functional derivative thereof comprises a point mutation in the amino acid sequence as defined in SEQ ID NO: 1, 4, 5 or 6.

A further aspect of the invention is to provide an isolated polynucleotide comprising a regulatory sequence of the ASMT gene or of the AANAT gene, characterized in that said regulatory sequence comprises an insertion/deletion mutation compared to the wild type regulate sequence of said ASMT gene or of said AANAT.

Another aspect of the invention concerns an isolated polypeptide encoded by a polynucleotide of the invention.

### BRIEF DESCRIPTION OF THE FIGURES

FIG. 1: Linkage and association of ASMT with ASD and ADHD. a. Linkage analysis of the PAR1 using 71 families with ASD. b. Linkage disequilibrium map of the ASMT gene. c. Association study of the ASMT gene in ASD and ADHD.

FIG. 2: Analysis of the ASMT mRNA in BLCL from ASD and controls. a. relative proportion of ASMT isoforms. Insert. ASMT isoforms from a control (C5), two ASD (A39 and A40), and cDNA from pineal gland. b. Relative abundance of ASMT correlated with rs4446909 genotype. c. Relative abundance of ASMT correlated with rs5989681 genotype. d. Promoter B sequence of the ASMT gene (SEQ ID NO:27 and SEQ ID NO:28). Rare and frequent variations identified in ASD are indicated in red and purple, respectively.

FIG. 3: Biochemical analyses in blood and BLCL from ASD, parents and controls. a. Serotonin concentration in the blood. b. ASMT activity in blood platelets. c. Melatonin concentration in the blood. d. ASMT activity in BLCL. C1 control age-matched for parents; C2 control age-matched for ASD.

FIG. 4: ASMT transcript level and activity in individuals with ASD and controls. ASD with GG and CG genotype at rs5989681 are in red and orange circle, respectively. Controls with GG and CG genotype at rs5989681 are in green and blue square, respectively. Individuals with low medium ASMT isoform and non-synonymous mutations are indicated by double lines and arrows, respectively.

FIG. 5: Position and segregation of the ASMT mutations in families with ASD, ADHD and anorexia.

FIG. 6: Melatonin concentration in the boy with ASD and their parents during the night. 0 is 20h30.

FIG. 7: Amino acid sequence of the HIOMT enzyme alternatively spliced with exon 6 and 7 (SEQ ID NO: 1; NCBI accession number AAA75290).

FIG. 8: Amino acid sequence of the HIOMT enzyme alternatively spliced with exon 7 and lacking exon 6 (SEQ ID NO: 2; NCBI accession number AAA75291).

FIG. 9: Amino acid sequence of the HIOMT enzyme lacking exon 6 and 7 (SEQ ID NO: 3; NCBI accession number AAA75289).

FIG. 10: Amino acid sequence of the AA.NAT enzyme (SEQ ID NO: 4; NCBI accession number NM_{—}001088).

FIG. 11: A. Localisation of the variations within the MTNR1A/MTNR1B receptors. B. Family with ADHD and the STOP mutation of MTNR1A. The variations changing amino acids highly or less conserved during evolution are indicated in red and orange respectively. The amino acids, which directly bind to melatonin are indicated in blue.

FIG. 12: Amino acid sequence of the melatonin MTNR1A receptor (SEQ ID NO: 5; NCBI accession number P48039).

FIG. 13: Amino acid sequence of the melatonin MTNR1B receptor (SEQ ID NO: 6; NCBI accession number AAS00461).

### DETAILED DESCRIPTION OF THE INVENTION

The present invention relates to methods for determining likelihood of an individual of developing a psychiatric disorder. The present invention further relates to compositions and methods for treating and/or preventing a psychiatric disorder such as autism, Deficits and Hyper Activity Disorder (ADHD) and anorexia.

### 1. Method of Diagnosis

According to an embodiment, the present invention provides a method for determining likelihood of an individual of developing a psychiatric disorder.

More specifically, the method of the invention is preferably achieved by identifying at least one genetic variation in a gene involved in the modulation of melatonin or in its mRNA transcript or by assaying the mRNA transcript level of said gene, or by assaying melatonin level.

As used herein, the expression “gene involved in the modulation of melatonin” refers to genes involved in the melatonin biosynthesis pathway and genes involved in the signalisation induced by the melatonin, such as its receptors.

Preferred susceptibility genes for psychiatric disorders contemplated by the method of the invention are ASMT (Acetyl serotonin methyl transferase) and AA-NAT (Arylalkylamine N. acetyl transferase) genes and genes that code for the melatonin receptors, such as the melatonin receptors MTNR1A and MTNR1B.

As used herein, the expression “genetic variation of a gene” refers to any variation that may occur within the DNA sequence of the genes contemplated by the present invention. Such a DNA sequence comprises, but is not limited to, regulatory sequences such as promoters, introns, exons, and coding sequences. By the term “genetic variation”, it is meant any variation that could interfere with the transcription and/or translation of a specific gene. Genetic variation may be recombination events or mutations such as substitution/deletion/insertion events like point and splice site mutations.

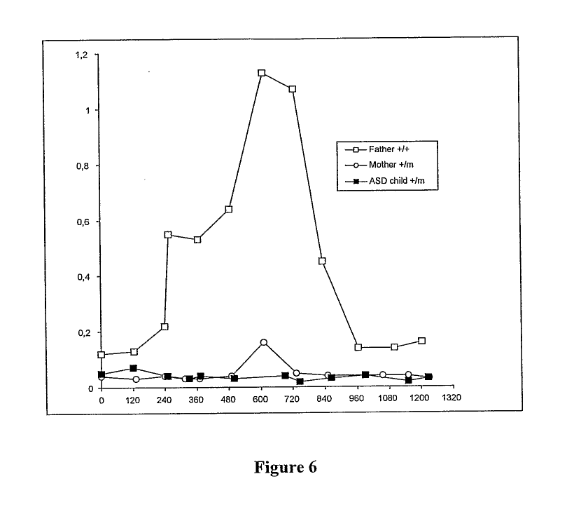

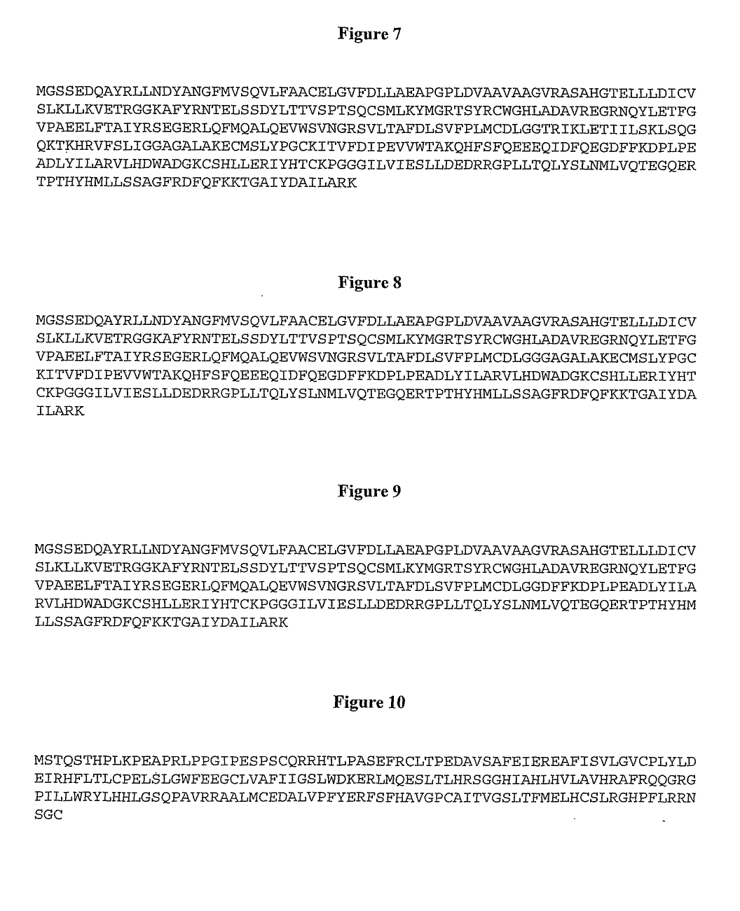

With respect to the ASMT gene, preferred genetic variations that the method of the invention is interested in, are the following preferred point mutations located at position 17, 81, 210, 306 or 326 as defined by the position in SEQ ID NO:1 (FIG. 7). More preferably the point mutation is selected from the group consisting of a N17K mutation, a K81E mutation, a R210H mutation, a G306A mutation and a L326F mutation. According to other preferred genetic variations regarding the ASMT gene concern point mutations located at position 17, 81, 228 or 298 as defined by the position in SEQ ID NO:2 (FIG. 8) or 17, 81, 231 or 251 as defined by the position in SEQ ID NO:3 (FIG. 9).

Another preferred genetic variation regarding the ASMT gene is a splice site mutation consisting of IVS5+2T>C.

Another preferred genetic variation regarding the ASMT gene is a mutation in a single nucleotide polymorphism (SNP) such as Rs5989681 or Rs6588809.

With respect to the AA.NAT gene, preferred genetic variations that the method of the invention is interested in, are the following preferred point mutations located at position 3, 13, 62, 157 or 163 as defined by the position in SEQ ID NO:4 (FIG. 10). More preferably the point mutation is selected from the group consisting of a T3M mutation, a A13S mutation, a V62I mutation, a A157V mutation and a A163V mutation.

With respect to a melatonin receptor encoding gene, preferred genetic variations that the method of the invention is interested in are those that occur preferably the genes coding for the melatonin receptors, such as MTNR1A (SEQ ID NO: 5) and MTNR1B (SEQ ID NO: 6). A preferred point mutation is a Y170X mutation in the MTNR1A protein as defined by the position in SEQ ID NO: 5 (FIG. 12).

According to another preferred embodiment, the method of the invention can be achieved by assaying the mRNA transcript level of a gene involved in the modulation of melatonin, such as those involved in its biosynthesis.

According to another preferred embodiment, the method of the invention can be achieved by assaying in a sample of the individual, the enzymatic activity of an enzyme involved in the melatonin biosynthesis pathway. Such an enzyme is preferably the HIOMT (Hydroxyindole-O-methyltransferase) enzyme. It will be understood that any suitable assay method known to one skilled in the art, such as the one described in Chanut et al.²⁸ and Finocchiaro et al.²⁹, is within the scope of the present invention.

According to another preferred embodiment, the method of the invention can be achieved by assaying in a sample of the individual, the melatonin level. Methods for assaying melatonin levels are known by one skilled in the art (see Tordjman et al.²⁵, Chanut et al.²⁸ and Finocchiaro et al.²⁹).

As used herein, the term “sample” refers to a variety of sample types obtained from an individual and can be used in accordance with the method of the invention. The definition encompasses blood, saliva, urine and other samples of biological origin.

As one skilled in that art may appreciate in view of the above, identification of a genetic variation and/or decrease in the enzymatic activity of said enzyme as compared to a psychiatric-free individual and/or a decrease in the transcript level of said gene, is indicative of a higher likelihood that the individual develops a psychiatric disorder.

### 2. Composition and Method of Use

According to another embodiment, the present invention concerns a composition for treating and/or preventing a psychiatric disorder in an individual having a defective gene involved in the modulation of melatonin. By the term “preventing” it refers to a process by which a psychiatric disorder is obstructed or delayed, whereas the term “treating” is intended, for the purposes of this invention, that the symptoms of the psychiatric disorder be ameliorated or completely eliminated.

As used herein, the term “defective gene” refers to a gene involved in the modulation of melatonin, such as those involved in its biosynthesis pathway, which undergoes genetic variation(s) that impairs or completely stops the production and/or the activity of the protein, such as an enzyme coded by the gene, thus interfering with the biosynthesis of melatonin.

More particularly, the composition of the invention comprises an acceptable carrier and a therapeutically effective amount of a molecule, preferably a polypeptide, such as an enzyme or functional derivatives thereof, promoting melatonin biosynthesis. Preferably, the enzyme is HIOMT (Hydroxyindole-O-methyltransferase) enzyme or functional derivatives thereof. A “functional derivative” of an enzyme, as is generally understood and used herein, refers to a protein/peptide sequence or fragment thereof or peptidomimetic thereof that possesses a functional biological activity that is substantially similar to the biological activity of the whole enzyme.

As used herein, the expression “an acceptable carrier” means a vehicle for containing the enzyme, preferably HIOMT, or functional derivatives thereof present in the composition of the invention that can be administered to an individual without adverse effects. Suitable carriers known in the art include, but are not limited to, gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

According to another embodiment, the present invention provides a method for treating and/or preventing a psychiatric disorder in an individual having a defective gene involved in the modulation of melatonin. In one embodiment, the method of the invention comprises the step of administrating in the individual a composition as defined above.

The amount of enzyme, preferably HIOMT, or functional derivatives thereof, present in the composition of the invention is preferably a therapeutically effective amount. A therapeutically effective amount of enzyme or functional derivatives thereof present in the composition of the invention is the amount necessary to allow the same to perform its role in the melatonin biosynthesis pathway without causing overly negative effects in the individual to which the composition is administered. The exact amount of enzyme or functional derivatives thereof present in the composition of the invention to be used and the composition to be administered will vary according to factors such as the type of psychiatric disorder being treated, the mode of administration, as well as the other ingredients in the composition.

The composition of the invention may be given to an individual through various routes of administration. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. Suitable dosages will vary, depending upon factors such as the amount of each of the components in the composition, the desired effect (short or long term), the route of administration, the age and the weight of the individual to be treated. Any other methods well known in the art may be used for administering the composition of the invention.

In another embodiment, the method for treating and/or preventing ASD in an individual comprises the step of administering in the individual a composition comprising melatonin or a functional derivative thereof.

### 3. Polynucleotides and Polypeptides of the Invention

A further embodiment of the invention concerns an isolated polynucleotide encoding a polypeptide or functional derivative thereof, characterized in that said polypeptide or functional derivative thereof comprises a point mutation in the amino acid sequence as defined in SEQ ID NO: 1, 4, 5 or 6.

Related aspects of the invention concern an isolated polynucleotide comprising a regulatory sequence of the ASMT gene or of the AANAT gene. The regulatory sequence comprises an insertion/deletion mutation compared to the wild type regulatory sequence of said ASMT or AANAT gene. Preferably, the regulatory sequence consists of a promotor.

According to another embodiment of the invention, there is provided an isolated polypeptide encoded by a polynucleotide as defined above.

As used herein, the term “isolated” is meant to describe a polynucleotide or a polypeptide that is in an environment different from that in which the polynucleotide or the polypeptide naturally occurs.

The present invention will be more readily understood by referring to the following example. This example is illustrative of the wide range of applicability of the present invention and is not intended to limit its scope. Modifications and variations can be made therein without departing from the spirit and scope of the invention. Although any methods and materials similar or equivalent to those described herein can be used in the practice for testing of the present invention, the preferred methods and materials are described.

### Example 1

### Mutations in the ASMT and AA-NAT Gene Decrease Melatonin Synthesis and Confer Susceptibility to Psychiatric Disorders

During the search of susceptibility factors to autism spectrum disorders (ASD) or Attention Deficits and Hyper Activity Disorder (ADHD), the inventors identified the ASMT (Acetyl serotonin methyl transferase) gene and the AA.NAT gene as a susceptibility gene for neuropsychiatric disorders. This gene is located on the pseudo autosomal region 1 (PAR1), common to the X and the Y chromosome¹⁴. ASMT encodes the enzyme HIOMT (Hydroxyindole-O-methyltransferase; EC 2.1.1.4), which catalyses the last step of melatonin biosynthesis^{15,16}.

Polymorphisms located in the promoter or the coding sequence and rare mutations affecting the splicing and the protein function are associated with a decrease of ASMT transcript level and/or activity. In individuals carrying these genetic variations, the decrease in ASMT activity leads to a decrease in melatonin concentration. Melatonin (N-acetyl-5-methoxytryptamine) is the major secretory product of the pineal gland. The synthesis of melatonin exhibits a pronounced circadian rhythm: its concentrations in the body are typically lower during the day and reach maximal levels at night in the darkness.

### Materials and Methods

### Mutation Screening and Genotyping

Both genotyping and screening for mutation was performed by direct sequencing. Coding regions including the exon/intron boundaries, the 5′ and 3′ untranslated regions, and the regulatory regions of the ASMT gene were amplified by PCR using HotstarTaq (Qiagen). For primers and PCR conditions, see table 8. Sequencing of PCR products was performed using the BigDye Terminator Cycle Sequencing Kit (V3.1, Applied Biosystems). Samples were then subjected to electrophoresis using an ABI PRISM genetic analyzer (Applied Biosystems). ABI electropherogram data were imported and analyzed for variation using the Genalys software (Author: Masazum Takahashi).

### RT-PCR and Quantitative RT-PCR

For analysis of ASMT transcripts from the investigated subjects, RNA was isolated from the generated lymphoblastoid cell lines using the NucleoSpin® RNA II kit (MACHEREY-NAGEL). Quantification of total RNA was performed by measurement of absorbance at OD₂₆₀ on a Biophotometer (Eppendorf). Oligo(dT) primed cDNA was prepared from 5 μg of this RNA using superscript II (Invitrogen) according to the manufacturers instructions in a reaction volume of 40 μl. For control of DNA contamination, a parallel tube without reverse transcriptase (RT negative control) was included in the RT reactions. All cDNA samples were tested on an agarose gel (3%) using a GAPDH PCR (for primers, see table 8), followed by a dilution with 40 to 80 μl dH20 (DEPC) in order to homogenize samples for real-time PCR analysis. The cDNA was used directly in TaqMan assays using the ABI PRISM 7500 Sequence Detection System (PE Biosystems). Each reaction was performed in triplicate and the cDNA was added to each reaction (25 μL) containing 1.25 μL of the assay and 12.5 μL of the TaqMan Universal PCR Master Mix (Applied Biosystems). Study samples were run in duplicate or triplicate on 96-well optical PCR plates (ABgene). Quantification of ASMT mRNA was performed using commercially available Assays-on-Demand. Two different assays were used, one covering the boundary between exon 1B and exon 2 (Hs00946625_m1), and one covering the boundary between exon 8 and exon 9 (Hs00187839_m1). Relative values of expression were determined for each sample using the standard curve method (ABI user's manual), and these values were normalized to the Ct values of GAPDH, a standard “housekeeping” control gene, using the glyceraldehyde phosphate dehydrogenase assay Hs99999905_m1. For ASMT and GAPDH, the thresholds were set at 0.2 and 0.25, respectively, which was within the linear region of the semi-log plot in all assays.

### Results

### Linkage Between the Pseudo Autosomal Region 1 and ASD

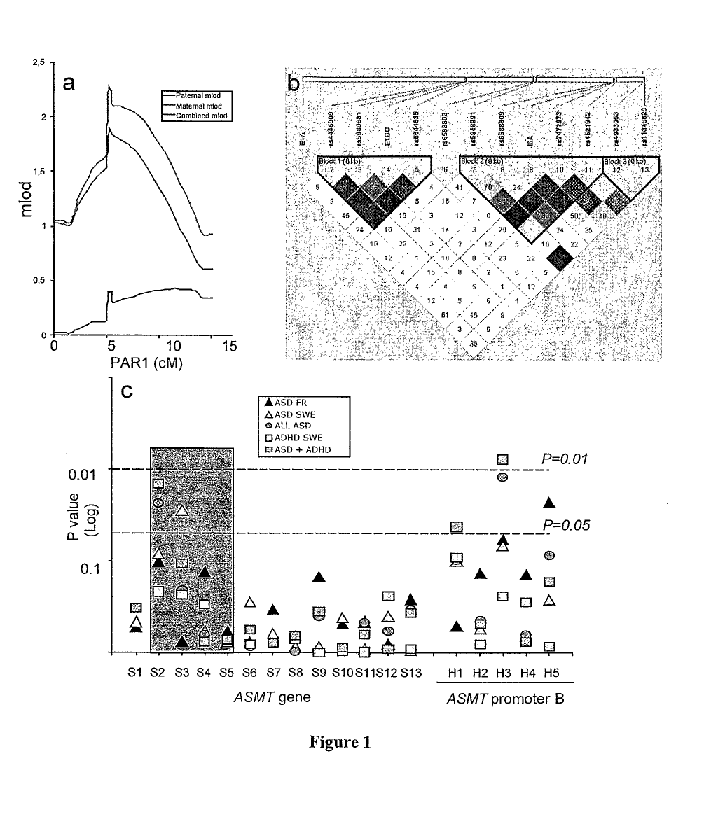

The Pseudo Autosomal Region 1 (PAR1) is only 2.7 Mb of DNA located on the tip of the short arms of the sex chromosomes and containing fifteen genes. A fine mapping of the PAR1 using four microsatellites markers (DXYS233, DXYS234, DXYS228 and DXYS229) was conducted on 52 families with at least two children with ASD. The inventors observed an excess of allelic sharing (Genehunter NPL=2.12; P=0.014 and ASPEX mlod=1.53). Within this region, the inventors focused their study on the ASMT gene using a combination of polymorphisms located in the promoters and intron 3 and adding 22 new families (FIG. 1a, Table 1). Consistent with the initial fine mapping results, the inventors observed an increased of ASMT allelic sharing in affected sib pairs (Genehunter NPL=2.95; P=0.0014 and ASPEX mlod=2.29; 0.0012). When families with only males affected sib pairs are used in the analysis, the linkage is dramatically increased (Genehunter NPL=4.8; P=4.86×10⁻⁷ and ASPEX mlod=5.92; P=1.81×10⁻⁷). However, in linkage studies of PAR1, the excess of paternal sharing should be taken with great care since this region is not fully independent from the sex chromosome segregation. This distortion effect specific to the paternal meiosis could account for the excess of sharing in male-male ASP and of non-sharing in male-female ASP. Nevertheless, the significant excess of maternal allelic sharing of ASMT in affected sib pairs (ASPEX maternal mlod=1.9; P=0.003) prompt the inventors to analyse the genetic variability of the ASMT gene in ASD compared to the control population.

### Genetic Variability of the ASMT Gene in ASD, ADHD and Controls

To determine whether frequent variations in ASMT significantly associate with ASD, the inventors genotyped one insertion/deletion located in the promoter A and twelve Single Nucleotide Polymorphisms (SNPs) in 275 ASD, 95 ADHD and 187 geographically-matched controls. The genotype region covers 41.3 kb, including the coding exons and the two promoters (FIG. 1b). The ASMT gene can be divided in five haplotype blocks of high inter-marker linkage disequilibrium (D′>0.8). Comparison of allelic, and genotype frequencies are presented in FIG. 1c and table 2. Overall, a significant association was observed between ASD and one SNP rs4446909 located in the promoter B (P=0.028). When the French and the Scandinavian samples are studied separately, a trend for association with rs4446909 is observed in the French sample (P=0.078) and a significant association with a close marker rs5989681 is present in the Scandinavian sample (P=0.047). The haplotype analysis indicated that one haplotype ACGC (including three SNPs in the promoter and one in the 5′UTR) was significantly less frequent in ASD (P=0.012) (Table 3). When populations are studied separately, although not significant, this haplotype was less frequent in both the French (P=0.057) and the Scandinavian ASD population (P=0.067) compared to their related control groups. In contrast, the GGGC haplotype could represent an “at risk haplotype” since it was more frequently observed in ASD (P=0.097). The same SNPs were genotyped in 95 Swedish individuals with ADHD, but no significant association was found. However, when individuals with ASD or ADHD were pooled and compared with controls, the association was more significant (rs4446909 P=0.015, ACGC, P=0.0077; GGGC, P=0.042).

### Correlation Between ASMT Genotype and Transcript Level

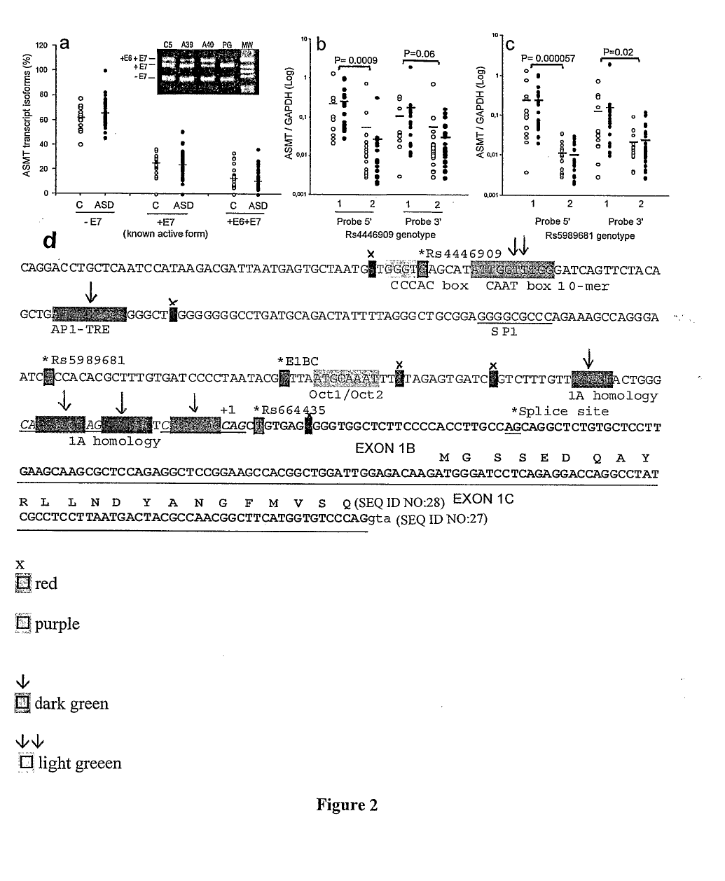

The ASMT gene contains two promoters (A and B) and two alternative spliced exons (Exon 6 and 7) (FIG. 2a). Promoter A was previously shown to be exclusive to retina ²⁷. Consistent with this, the inventors could not amplify ASMT transcripts originated from promoter A using human B lymphoblastoid cell lines (BLCL) or pineal gland cDNA. In contrast, the inventors could detect the three known alternative isoforms derived from promoter B (FIG. 2b insert). The long isoform contains all exons including exon 6, which is a Long Interspersed Nuclear Element (LINE). The medium isoform does not contain the LINE insertion and codes for the isoform homologous to the ASMT protein of other species. The shortest isoform does not contain the LINE insertion and exon 7. Only the medium isoform is supposed to be functional since the insertion of exon 6 or the deletion of exon 7 greatly modify the O-methylase domain of ASMT. Using fluorescent RT-PCR, the inventors could ascertain the relative proportion of the three ASMT isoforms. Overall, there was no significant difference in the abundance of the three isoforms between ASD and controls (FIG. 2b). However, in several individuals (13/48 patients and 4/23 controls), the relative percentage of the medium isoform was relatively low (<20%). For example, in the individual ASD49, the long ASMT isoform including the LINE was repetitively more abundant than the medium isoform (FIG. 2b insert). Interestingly, one non synonymous SNP rs6588809 (R190W), located in exon6 within the LINE element, was associated with the level of LINE insertion (P=0.004). In average, individuals homozygotes for the C allele had 3.6 fold more insertion of the LINE in the ASMT transcript compared to individuals homozygotes for the T allele. This variation rs6588809 is probably located in one exonic splicing enhancer (ESE), a sequence which binds to splicing factors such as SR proteins. Despite, its functional consequence on the relative abundance of ASMT isoforms, no association was observed between rs6588809 and ASD.

To quantify the level of ASMT transcripts, the TaqMan technology and two independent probes E1 CE2 and E8E9 located in the 5′ and the 3′ end of the mRNA respectively were used. No significant difference in ASMT mRNA level was observed between our sample of 60 ASD and 35 control individuals (FIG. 2c). In both samples, the ASMT transcript level was heterogenous ranging from 0.0019 to 1.39 compared to GAPDH mRNA level. However, when the ASMT genotypes and transcript levels were compared, the inventors could observe a very significant association between the transcript level and two SNPs rs4446909 (5′ Probe P=0.0009; 3′ probe P=0.06) and rs5989681 (5′ probe P=0.000057; 3′probe P=0.02). These SNPs are distant from 109 bp, in high linkage disequilibrium (D′=0.94) and located in promoter B (FIG. 2d). The SNP rs4446909 is situated at −207 bp from the transcription site in a CCCAC box and six nucleotides downstream a CAAT/10 mer box also present in the promoter A (FIG. 2d). SNP rs5989681 is located at −97 bp from the transcription site in a putative binding site for the transcription factor NF-kappaB. Individuals homozygote for the G allele of both SNPs had less ASMT transcripts compared to heterozygotes individuals. Two individuals with a rs4446909 G/G genotype and a rs5989681 C/G genotype had a high transcript level, suggesting that the C allele of rs5989681 could be responsible for the high transcript level. Interestingly, these two SNPs (rs4446909 and rs5989681) were the one associated with ASD. The G alleles of both SNPs were more frequent in ASD and ADHD and were associated to a low ASMT transcript level. To explore further the relation between the ASMT gene and the susceptibility to these disorders, the inventors measured the enzyme activity in families with ASD and controls.

### ASMT Activity in ASD and Control Individuals.

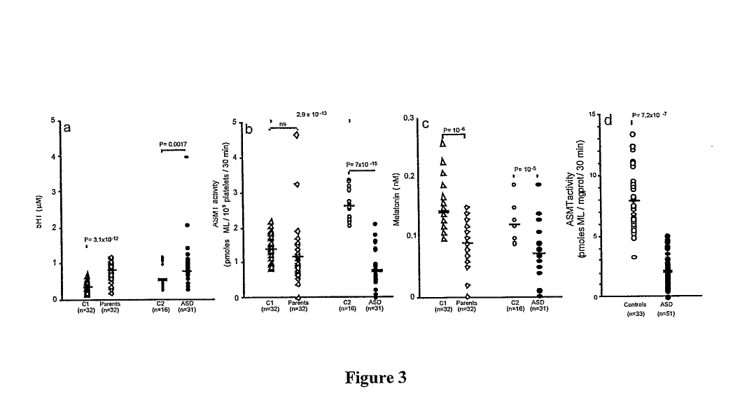

The ASMT activity was first investigated in the blood platelets of ASD, their parents and control individuals. As already reported in other samples, the inventors could observe an increase of serotonin concentration in the blood of ASD (5HT 1.0±0.65 μM; P=1.46×10⁻⁷) and their parents (5HT 0.80±0.24 μM; P=1.85×10⁻¹⁰) compared to controls (5HT: 0.42±0.22 μM; FIG. 3a). Remarkably, the inventors also observed a strong decrease of ASMT activity for almost all of the individuals with ASD (ASMT blood 0.77±0.47 pmoles/10⁹ platelets/30 min P=1.15×10⁻¹⁰) and their parents (1.18±0.87 pmoles/10⁹ platelets/30 min P=0.00045) compared to control (ASMT blood 1.811±0.68 pmoles/10⁹ platelets/30 min; FIG. 3b). In parents, the ASMT activity was significantly decreased in the mothers (P=0.016), but not in the fathers (P=0.80) compared to sex- and age-matched controls. The decrease in ASMT was accompanied by a significant decrease of blood melatonin concentration in ASD (ML 0.07±0.04 nM; P=5.2×10⁻¹⁰) and their parents (ML 0.09±0.04 nM; P=2.04×10⁻⁶) compared to controls (ML 0.14±0.04 nM) (FIG. 3C). In parents, the melatonin concentration was significantly lower in mother (P=3.35×10−5) and in the fathers (P=0.007). To replicate these results, BLCLs from 51 individuals with ASD (11 already analysed in the blood sample) and 33 new independent controls were analysed (FIG. 3d). Consistent, with the results obtained using the blood platelets, the level of ASMT activity was dramatically decreased in ASD (ASMT BLCL 3.29±2.5 pmoles/mg prot/30 min) compared to controls (ASMT BLCL 7.97±2.5 pmoles/mg prot/30 min; P=7.2×10⁻⁷).

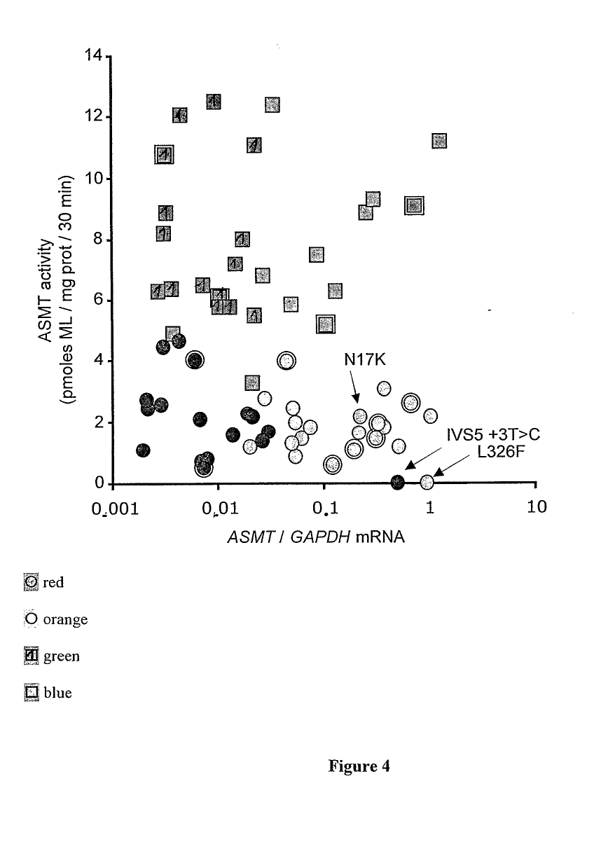

The inventors next plotted the relative quantity of ASMT mRNA against the level of the enzyme activity (FIG. 4). In the control sample, the inventors could not detect a significant correlation between the RNA amount and the enzyme activity, suggesting that the level of ASMT transcript is not a strong limiting factor for enzyme activity in BLCL. In the ASD sample, the ASMT activity was decreased in almost all of the individuals, independently of their transcript level. For several individuals with ASD, the inventors cannot exclude that the low ASMT activity could be the direct consequence of a very low ASMT transcript level and/or a specific strong decrease in the medium isoform. Nevertheless, these results indicated that the global decrease of ASMT activity observed in ASD could not be explained solely by a decrease in ASMT mRNA. To further investigate this enzymatic deficiency, we screen the ASMT gene for mutation.

### Mutation Screening of the ASMT Gene in ASD, ADHD and Controls

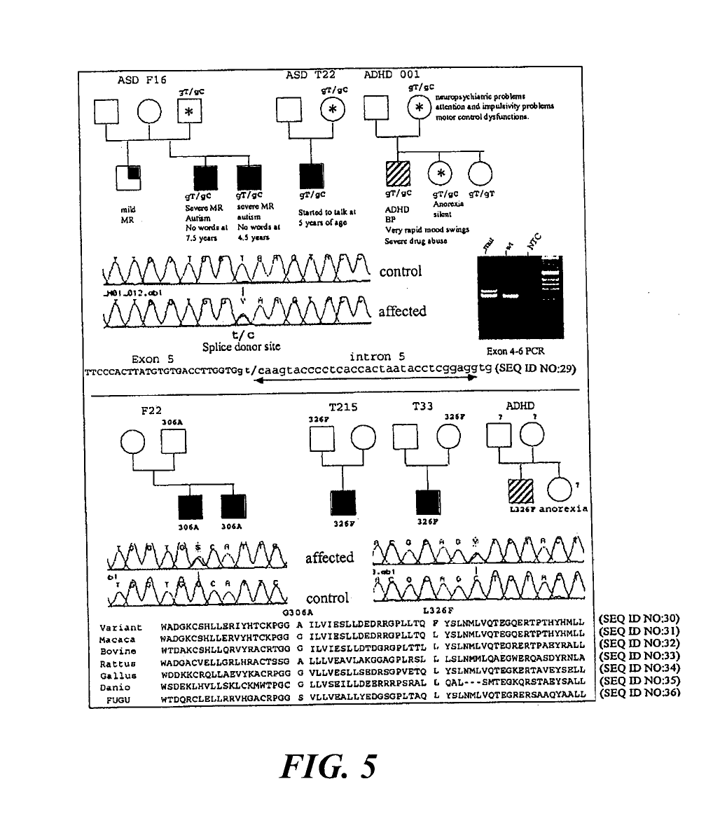

All exons of the ASMT gene, including the two promoters and alternatively spliced exons were directly sequenced in individuals with ASD (n=275) or ADHD (n=103). Several genetic variants were identified including a splice site mutation (IVS5+2T>C), five rare non-synonymous variations (N17K, K81E, R210H, G306A, L326F) and two synonymous variation (N167N, Q205Q). The location, the segregation and the frequency of these rare variations are indicated in FIG. 5 and table 4. The splice site mutation (IVS5+2T>C) was present in two families with ASD and one with ADHD, but never observed in 411 controls. In one patient carrying the splice site mutation (IVS5+2T>C), the inventors could detect additional transcripts isoforms compared to controls, which originate from a donor splice site located 31 bp in intron 5 (FIG. 5). This abnormal spliced transcripts leads to different C-terminal protein sequences after amino acid G188 and premature truncation of all ASMT isoforms (FIG. 5). In ASD family 1, the mother transmitted the mutation to her son, who was homozygote for the G alleles of rs4446909 and rs5989681. In ASD family 2, the father transmitted the splice site mutation to his two sons with autism. The mother had previously a first child with autism but with a milder phenotype and from a different father. The haplotype analysis showed that the mother has transmitted the same ASMT GGGT promoter haplotype to all her affected sons. In the ADHD family 1, the mother carrying the splice site mutation had history of neuropsychiatric disorders including attention and impulsivity problems. She transmitted the mutation to her son with ADHD and to her daughter with anorexia, but not to her unaffected daughter. All individuals carried the haplotype. The N17K variation was observed in two ASD families (family 3 and 4) with parents originated from Asiatic countries. In family 3 and 4, the mother transmitted the mutation. In ASD family 5, the mother transmitted the non-synonymous variation K81E. In ASD family 6 from Norway, the father transmitted a variation G306A to his two sons with autism. The variation L326F was identified in two independent families with ASD and one with ADHD. In family 7 and 8, the mutations were transmitted by the mother and the father, respectively. In ADHD family 2, the son with ADHD has a 326 mutation and there is a daughter with anorexia. None of these non-synonymous variations were observed in controls except L326F, which was present in 3/400 controls. To ascertain the functional effects of these rare variants, the inventors measured the ASMT enzyme activity and the melatonin concentration in the carriers.

### Biochemical and Clinical Characterisation of the Individuals Carrying Rare Variations of the ASMT Gene.

Biochemical analyses of the blood platelets indicate that individuals carrying the splice site mutation (IVS5+2T>C) or the L326F mutation have low HIOMT activity, a decrease of melatonin and a relatively high concentration of 5-HT compared to controls (Table 5). Consistent with the results obtained on the blood samples, ASMT activity in BLCL was found reduced in all individuals carrying the (IVS5+2T>C) and the L326F mutations. In these individuals, the melatonin concentration was also found decreased compared to controls. To further explore the ASMT deficit in vivo, the inventors investigated family 1 carrying the splice site mutation (FIG. 6). The father does not carry the mutation and showed a normal increase of melatonin during the night. In contrast, both carriers of the ASMT splice mutation, the unaffected mother and the son with ASD, showed no increase of melatonin during the night. Despite this absence of melatonin, no obvious difference in the sleep pattern was observed compared to controls.

Mutations of AANAT in Individuals with ASD and ADHD

In the melatonin biosynthesis pathway, the enzyme AANAT is located before the ASMT and transform the serotonin into N-acetyl serotonin. Mutations in the AANAT gene were screened and five rare non-synonymous variations (T3M, A13S, V621, A157V, A163V) were indetified in individuals with ASD and ADHD. These variations were not observed in 200 controls.

### Example 2

### Mutations in the Melatonin Receptors MTNR1A and MTNR1B Decrease Melatonin Binding and Confer Susceptibility to Psychiatric Disorders

During the search of susceptibility factors to autism or Attention Deficits and Hyper Activity Disorder (ADHD), the inventors identified genetic mutations in the ASMT (Acetyl serotonin methyl transferase) gene (see Example 1). In this present example, the inventors report genetic variations in the melatonin receptors including a stop mutation in a patient with ADHD, which is absent from all controls tested (n=150). In addition, the inventors identified an ASMT splice site mutation in a patient with OCD. These results confirm the results of Example 1 showing that the melatonin pathway is associated to psychiatric disorder.

### Results

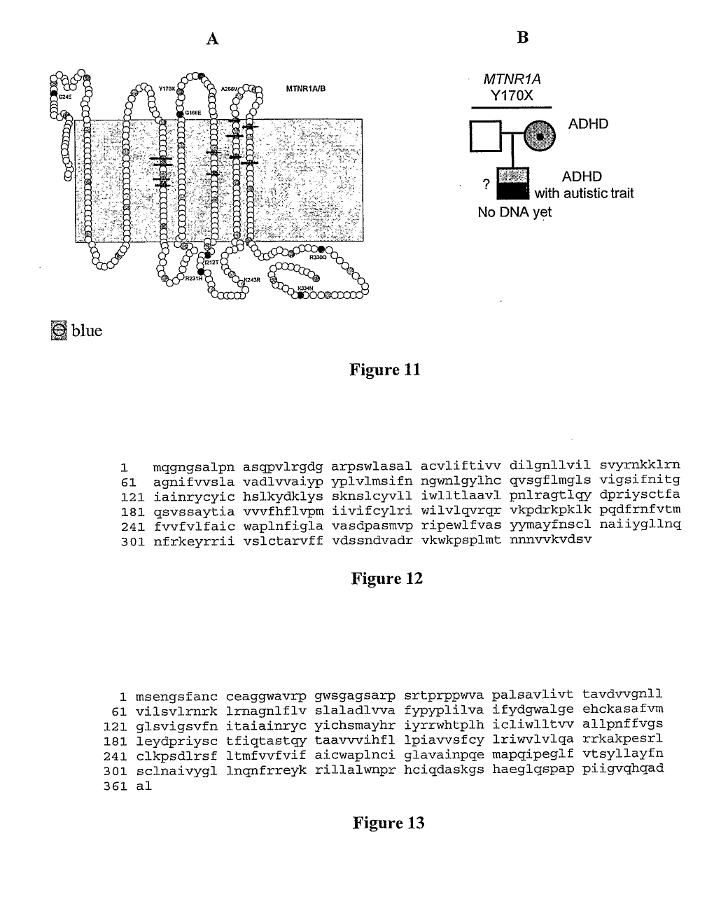

The inventors investigated whether variations in MTNR1A and MTNR1B were associated with ASD and ADHD, by directly sequencing all exons and the promoters of individuals with ASD (n=78-130) or ADHD (n=79-102). Several exonic mutations were identified (Table 9, FIG. 11A), including a stop mutation in a patient with ADHD, caused by a point mutation at position 170 (see SEQ ID NO: 5), namely a Y170X mutation. This woman has a son suffering from ADHD with autistic traits, but no DNA is available for this patient.

On the basis of the results shown in Example 1, the inventors have now evidenced that the melatonin receptor MTNR1A can also be mutated in individuals with ADHD. These results confirm that abnormal melatonin pathway is a risk factor for neuropsychiatric disorders.

### General Conclusions

Melatonin is synthesised in two steps from the neurotransmitter serotonin. The HIOMT enzyme catalyses the final reaction transforming N-acetylserotonin into melatonin. This hormone is mainly produced in the pineal gland but also in other tissues such as retina or lymphocytes. It is secreted during the circadian cycle with high level during the night and low level during the day. The physiological role of melatonin is still unclear but it plays crucial roles correlated to the circadian or seasonal rhythms. Furthermore, melatonin was shown to have anti-oxidative or anti-aging properties as well as effects on hormonal autocrine/paracrine functions, on the immune system, on the modulation of neurotransmitter release.

Sleep problems are frequently reported in patients with neuro-psychiatric disorders. In addition, an increase concentration of serotonin in individuals with autism as well as in there relatives is one of the most replicated finding in autism. However, despite numerous genetic studies on the serotonin transporters or receptors, the reason why there is high level of serotonin was never elucidated. The outcomes of the present invention are extremely important for the diagnostic and therapy of autism and ADHD for at least three major reasons:

A. The biochemical analysis of the melatonin biosynthesis is the first biological marker associated with susceptibility to neuropsychiatric disorders (autism, ADHD and OCD). This assay could be used as a screening test since concentration of melatonin could be quantified with a simple saliva sample and the HIOMT enzymatic activity can be measured using a blood sample.
B. In the case of a decrease melatonin concentration is identified in one patient, the supplementation with exogenous melatonin is feasible and could correct the genetic deficit.
C. The identification of these mutations should permit to find others susceptibility factors having roles in the same biological pathway.

TABLE 1
ASMT allelic sharing in ASD sib-pairs families

| Affected | IBD status | | | | | Parental sharing % (IBD1:IBD0)* | | | |
|---|---|---|---|---|---|---|---|---|---|
| sib pairs | 0 | M1 | P1 | 2 | P value | Maternal | P value | Paternal | P value |
| Male-Male | 3 | 4 | 5 | 15 | 0.0033 | 71 (20:8) | 0.023 | 75 (24:8) | 0.0047 |
| Male-Female | 8 | 8 | 1 | 5 | 0.11 | 60 (15:10) | 0.31 | 31 (6:18) | 0.014 |
| Female-Female | 1 | 0 | 1 | 1 | — | 33 (1:2) | — | 75 (3:1) | — |
| All | 12 | 12 | 7 | 21 | 0.049 | 64 (36:20) | 0.033 | 62 (33:27) | 0.44 |

*Thirteen additional families. informative for only one of the parent. were included in the parental sharing compare to the IBD status.

TABLE 2
Association study of ASMT frequent variations identified in ASD, ADHD and controls.

| | | | | | ASD | | | | | | | | | | |
|---|---|---|---|---|---|---|---|---|---|---|---|---|---|---|---|
| | | Geno- | ASD | C | FR P | ASD | ADHD | C | ASD | P | All | ASD + | ALL | P | P |
| SNP | Localisation | type | FR | FR | value | SWE | SWE | SWE | P value | value | ASD | ADHD | C | value | value |
| EIA | 1757953 | A | 0.644 | 0.600 | 0.52 | 0.637 | nd | 0.581 | 0.46 | | | | | 0.32 | |
| | | A:A | 20 | 12 | | 17 | nd | 14 | | | 37 | | 26 | | |
| | | A:G | 27 | 30 | 0.24 | 17 | nd | 22 | 0.64 | | 44 | | 52 | 0.28 | |
| | | G:G | 3 | 3 | | 6 | nd | 7 | | | 11 | | 10 | | |
| rs4446909 | 1977610 | A | 0.234 | 0.309 | 0.078 | 0.212 | 0.222 | 0.280 | 0.14 | 0.21 | | | | 0.028 | 0.017 |
| | | A:A | 8 | 6 | | 4 | 1 | 9 | | | 12 | 13 | 15 | | |
| | | A:G | 57 | 38 | 0.17 | 28 | 33 | 33 | 0.32 | 0.056 | 85 | 118 | 71 | 0.09 | 0.039 |
| | | G:G | 91 | 37 | | 53 | 45 | 49 | | | 144 | 189 | 86 | | |
| rs5989681 | 1777719 | C | 0.338 | 0.358 | 0.65 | 0.230 | 0.265 | 0.324 | 0.047 | 0.23 | | | | 0.21 | 0.1 [symbol] |
| | | C:C | 17 | 9 | | 5 | 2 | 10 | | | 22 | 24 | 19 | | |
| | | C:G | 72 | 40 | 0.84 | 30 | 30 | 39 | 0.15 | 0.089 | 107 | 141 | 79 | 0.44 | 0.24 |
| | | G:G | 68 | 32 | | 52 | 40 | 42 | | | 120 | 160 | 74 | | |
| EIBC | 1777747 | A | 0.101 | 0.062 | 0.14 | 0.098 | 0.086 | 0.121 | 0.48 | 0.29 | | | | 0.74 | 0.85 |
| | | A:A | 3 | 1 | | 1 | 1 | 0 | | | 4 | 5 | 1 | | |
| | | A:G | 26 | 8 | 0.35 | 15 | 12 | 22 | 0.32 | 0.18 | 41 | 53 | 30 | 0.62 | 0.62 |
| | | G:G | 129 | 72 | | 71 | 68 | 69 | | | 200 | 268 | 141 | | |
| rs6644635 | 1777821 | C | 0.661 | 0.648 | 0.77 | 0.622 | 0.619 | 0.682 | 0.85 | 0.74 | | | | 0.81 | 0.79 |
| | | C:C | 68 | 32 | | 32 | 32 | 52 | | | 100 | [symbol] | 64 | | |
| | | C:T | 7 | 11 | 0.81 | 43 | 45 | 51 | 0.6 | 0.92 | [symbol] | 161 | [symbol] | 0.46 | 0.64 |
| | | T:T | 17 | 8 | | 11 | 7 | 8 | | | 28 | [symbol] | 116 | | |
| rs [symbol] 588802 | 1785774 | C | 0.593 | 0.568 | 0.62 | 0.425 | 0.476 | 0.491 | 0.29 | 0.82 | | | | 0.96 | 0.66 |
| | | C:C | 58 | 19 | | 20 | 20 | 11 | | | 78 | 98 | 30 | | |
| | | C:T | 62 | 37 | 0.13 | 34 | 40 | 30 | 0.096 | 0.58 | 96 | 136 | 67 | 0.018 | 0.023 |
| | | T:T | 30 | 10 | | 33 | 24 | 12 | | | 63 | 87 | 22 | | |
| rs5948991 | 178616 [symbol] | C | 0.163 | 0.210 | 0.34 | 0.141 | 0.000 | 0.118 | 0.61 | nd | | | | 0.78 | nd |
| | | C:C | 2 | 5 | | 1 | nd | 3 | | | 3 | | 8 | | |
| | | C:T | 13 | 27 | 0.65 | 9 | nd | 16 | 0.73 | nd | 22 | | 43 | 0.9 | nd |
| | | T:T | 37 | 56 | | 29 | nd | 74 | | | 66 | | 130 | | |
| rs6588809 | 1795429 | C | 0.491 | 0.512 | 0.65 | 0.474 | 0.433 | 0.453 | 0.69 | 0.7 | | | | 0.93 | 0.76 |
| | | C:C | 45 | 19 | | 22 | 17 | 16 | | | 67 | 84 | 35 | | |
| | | C:T | 75 | 44 | 0.28 | 47 | 44 | 46 | 0.74 | 0.79 | 122 | 166 | 90 | 0.27 | 0.29 |
| | | T:T | 48 | 17 | | 27 | 29 | 24 | | | 75 | 104 | 41 | | |
| I6A | 1793531 | A | 0.494 | 0.425 | 0.15 | 0.484 | 0.494 | 0.494 | 0.83 | 0.99 | | | | 0.39 | 0.36 |
| | | A:A | 39 | 15 | | 25 | 24 | 19 | | | 84 | 88 | 34 | | |
| | | A:G | 88 | 38 | 0.29 | 43 | 41 | 27 | 0.41 | 0.48 | 131 | 172 | 85 | 0.65 | 0.55 |
| | | G:G | 41 | 27 | | 28 | 25 | 20 | | | 69 | 94 | 47 | | |
| rs7471973 | 1795551 | C | 0.814 | 0.787 | 0.48 | 0.802 | 0.833 | 0.831 | 0.47 | 0.96 | | | | 0.99 | 0.83 |
| | | C:C | 109 | 48 | | 63 | 63 | 57 | | | 172 | 235 | 105 | | |
| | | C:T | 54 | 30 | 0.72 | 28 | 24 | 29 | 0.091 | 0.16 | 82 | 106 | 59 | 0.27 | 0.19 |
| | | T:T | 4 | 2 | | 5 | 3 | 0 | | | 9 | 12 | 2 | | |
| rs4521942 | 1795844 | G | 0.913 | 0.930 | 0.49 | 0949 | 0.944 | 0.944 | 0.83 | 0.98 | | | | 0.54 | 0.7 |
| | | G:G | 139 | 80 | | 90 | 80 | 80 | | | 229 | 309 | 160 | | |
| | | G:T | 27 | 13 | 0.67 | 8 | 8 | 10 | 0.5 | 0.54 | 35 | 43 | 23 | 0.49 | 0.46 |
| | | T:T | 1 | 0 | | 1 | 1 | 0 | | | 2 | 3 | 0 | | |
| rs4933063 | 1799231 | C | 0.910 | 0.914 | 0.86 | 0.872 | 0.930 | 0.896 | 0.48 | 0.2 | | | | 0.67 | 1 |
| | | C:C | 147 | 79 | | 75 | 80 | 73 | | | 222 | 302 | 152 | | |
| | | C:T | 28 | 12 | 0.67 | 21 | 13 | 17 | 0.77 | 0.36 | 49 | 62 | 29 | 0.84 | 0.83 |
| | | T:T | 2 | 2 | | 2 | 0 | 1 | | | 4 | 4 | 3 | | |
| rs11346829 | 1799288 | A | 0.116 | 0.086 | 0.28 | 0.097 | 0.102 | 0.099 | 0.95 | 0.92 | | | | 0.41 | 0.44 |
| | | A:A | 2 | 0 | | 2 | 2 | 2 | | | 4 | 6 | 2 | | |
| | | A:G | 37 | 16 | 0.44 | 15 | 15 | 14 | 0.99 | 0.99 | 52 | 67 | 30 | 0.72 | 0.74 |
| | | G:G | 138 | 77 | | 81 | 76 | 75 | | | 219 | 295 | 152 | | |

[symbol]

indicates data missing or illegible when filed

TABLE 3
Haplotypic association using the four frequent polymorphisms located in the
promoter B and 5′UT5 of ASMT

| | French sample | | | | | | | | | |
|---|---|---|---|---|---|---|---|---|---|---|
| | (%) | | | Scandinavian sample (%) | | | | | Combined sample | |
| | | | P | | | | ASD | ADHD | ASD | ASD + ADHD |
| | ASD | Control | values | ASD | ADHD | Controls | P value | Pvalue | P value | P value |
| GGGC | 0.308 | 0.279 | 0.51 | 0.396 | 0.374 | 0.313 | 0.099 | 0.058 | 0.096 | 0.042 |
| ACGC | 0.225 | 0.304 | 0.057 | 0.185 | 0.214 | 0.267 | 0.067 | 0.24 | 0.012 | 0.0077 |
| GGGT | 0.242 | 0.306 | 0.13 | 0.272 | 0.267 | 0.241 | 0.54 | 0.90 | 0.43 | 0.47 |
| GCGC | 0.112 | 0.052 | 0.022 | 0.017 | 0.051 | 0.037 | 0.26 | 0.82 | 0.086 | 0.17 |
| GGAT | 0.092 | 0.054 | 0.14 | 0.098 | 0.078 | 0.108 | 0.75 | 0.25 | 0.68 | 0.76 |
| GCGT | 0 | 0 | — | 0.013 | 0 | 0.013 | 0.99 | — | — | — |
| AGGC | 0 | 0 | — | 0.018 | 0 | 0.011 | 0.59 | — | 0.32 | — |

TABLE 4
ASMT Rare variations identified in ASD, ADHD and controls.

| | | | French | Swedish | Swedish | French | Swedish | [symbol] th Africa | |
|---|---|---|---|---|---|---|---|---|---|
| Exon/Intron | Variant | Nucleotide* | ASD | ASD | ADHD | Control | Control | Control | All controls |
| 5′ UTR | | C/T | 0\|136 | 0\|82 | 0\|103 | 0\|84 | 1\|90 | Nd | 1/174 |
| E1A | | A/g | 2\|136 | 0\|82 | 0\|103 | 0\|84 | 0\|90 | Nd | 0/174 |
| E1A | | G/C | 2\|136 | 0\|82 | 0\|103 | 0\|84 | 0\|90 | Nd | 0/174 |
| E1A | | A/T | 2\|136 | 0\|82 | 0\|103 | 0\|84 | 0\|90 | Nd | 0/174 |
| E1A | | A/g | 2\|136 | 0\|82 | 0\|103 | 0\|84 | 0\|90 | Nd | 0/174 |
| E1A | | C/T | 1\|136 | 0\|82 | 0\|103 | 0\|84 | 0\|90 | Nd | 0/174 |
| E1A | | C/A | 0\|136 | 1\|82 | 0\|103 | 0\|84 | 0\|90 | Nd | 0/174 |
| E1B | N17K | C232A | 1\|140 | 1\|80 | 0\|103 | 0\|79 | 0\|92 | Nd | 0/171 |
| E1B | | A/T | 1\|140 | 0\|80 | 0\|103 | 0\|79 | 0\|92 | Nd | 0/171 |
| E1B | | C/T | 2\|140 | 0\|80 | 0\|103 | 0\|79 | 0\|92 | Nd | 0/171 |
| E1B | | C/T | 1\|140 | 1\|80 | 0\|103 | 0\|79 | 0\|92 | Nd | 0/171 |
| E1B | | C/A | 0\|140 | 1\|80 | 0\|103 | 0\|79 | 0\|92 | Nd | 0/171 |
| E2 | K81E | A422G | 1\|150 | 0\|86 | 0\|103 | 0\|67 | 0\|53 | Nd | 0/120 |
| INTRON 2 | | C/A | 1\|150 | 0\|86 | 0\|103 | 0\|67 | 0\|53 | Nd | 0/120 |
| INTRON 2 | | G/A | 3\|150 | 0\|86 | 0\|103 | 0\|67 | 0\|53 | Nd | 0/120 |
| INTRON 4 | | C/T | 1\|174 | 0\|97 | 0\|103 | 0\|94 | 0\|93 | Nd | 0/187 |
| E5 | N167N | | 0\|182 | 1\|102 | 0\|103 | 0\|94 | 0\|145 | 0\|172 | 0/411 |
| INTRON 5 | IVS5 + 2T > C | T/C | 2\|182 | 0\|102 | 1\|102 | 0\|94 | 0\|145 | 0\|172 | 0/411 |
| INTRON 6 | | G/A | 1\|168 | 0\|95 | 0\|103 | 0\|80 | 0\|87 | Nd | 0/167 |
| E7 | D238G | A894G | 0\|166 | 0\|97 | 0\|103 | 0\|94 | 1\|97 | Nd | 1/191 |
| E7 | K247R | A921G | 0\|166 | 0\|97 | 0\|103 | 1\|94 | 0\|97 | Nd | 1/191 |
| INTRON 7 | | C/T | 0\|166 | 0\|97 | 0\|103 | 0\|94 | 1\|97 | Nd | 1/191 |
| E8 | P271L | G994T | 0\|176 | 0\|96 | 0\|103 | 0\|94 | 1\|93 | Nd | 1/187 |
| E8 | C301S | G1083C | 0\|176 | 0\|96 | 0\|103 | 1\|94 | 0\|93 | Nd | 1/187 |
| E9 | G306A | G1098C | 0\|186 | 1\|101 | 0\|103 | 0\|88 | 0\|145 | 0\|95 | 0/328 |
| | R319Q | G1137A | 0\|186 | 0\|101 | 0\|103 | 1\|88 | 0\|145 | 0\|95 | 0/328 |
| | L326F | C1157T | 1\|186 | 1\|101 | 1\|103 | 0\|88 | 2\|145 | 1\|95 | 3/328 |

*reference sequence Genebank NM004043 [symbol]
indicates data missing or illegible when filed

TABLE 5
Biochemical measurement in blood and BLCL from families carrying rare variations

| | | | Blood sample | | | BLCL | |
|---|---|---|---|---|---|---|---|
| | | | | HIOMT | | HIOMT | |
| | Axe I | ASMT | 5-HT | (pmoles/10⁹ | ML | (pmoles/ | ML |
| | Diagnosis | genotype | (μM) | platelets/30 min) | (nM) | mg proi/30 min) | (pM) |
| Father | Nd | +/+ | 1.14 | 0.96 | 0.12 | — | |
| Mother | Nd | +/(IVS5 + 2T > | 1.18 | 0.39 | 0.05 | — | |
| | | C) | | | | | |
| Proband 1 | Autism | +/(IVS5 + 2T > | 0.89 | 0.22 | 0.04 | <0.01 | <0.02 |
| | | C) | | | | | |
| Father | Nd | +/+ | 0.90 | 0.88 | 0.09 | — | — |
| Mother | Nd | +/L326F | 0.96 | <0.06 | <0.02 | — | — |
| Proband 2 | Autism | +/L326F | 0.80 | <0.06 | <0.02 | <0.01 | <0.02 |
| Controls | | | 0.12-1.17 | 0.86-3.36 | 0.1-0.26 | 3.3-13.4 | 2.3-10 |
| range (n = 47) | | | | | | | |

TABLE 6
Genetic variations of the AANAT gene identified
in individuals with ASD, ADHD and controls.

| | | ASD | ADHD | Controls |
|---|---|---|---|---|
| | Localisation* | (n = 287) | (n = 96) | (n = 165) |
| Promoter | TG | 169:179 | | 170:164 |
| | G | 179:169 | | 180:140 |
| 5′UTR | | | | |
| Intron 1 | | | | |
| Exon2 | C242T | 3 | | 0 |
| | (T3M) | | | |
| | G271T | 1 | | 0 |
| | (A13S) | | | |
| Intron 2 | GA | | | |
| Exon3 | G418A | 1 | 1 | 0 |
| | (V62I) | | | |
| Intron 3 | | | | |
| Exon 4 | TC (H122H) | 1 | 0 | 0 |
| | CT (R128R) | 1 | 0 | 0 |
| | C689T | 0 | 1 | 0 |
| | (A152V) | | | |
| | CT (A156A) | 0 | 1 | 0 |
| | C704T | 1 | 0 | 0 |
| | (A157V) | | | |
| | C722T | 1 | 1 | 0 |
| | (A163V) | | | |
| | GA (G177D) | 0 | 0 | 1 |
| | CA (I181I) | 1 | 0 | 0 |
| 3′UTR | CT | | | |
| | GA | | | |

*reference sequence Genebank NM_001088

TABLE 7
Allelic and haplotypic association using two AANAT frequent
promoter polymorphisms

| | | | Swedish | | | |
|---|---|---|---|---|---|---|
| | Feench | | ADHD | | | |
| | and | | n = 92 | | | |
| | Swedish | | Major Allele | | French | Swedish |
| | ASD | P | Frequence | P | Controls | controls |
| | N = 192 | value | P value | value | n = 95 | N = 95 |
| Single | | | | | | |
| rs | G 0.51 | 0.54 | T 0.57 | 0.45 | G 0.512 | T 0.53 |
| rs | G 0.51 | 0.21 | G 0.57 | 0.45 | G 0.596 | G 0.53 |
| Haplo- | | | | | | |
| type | | | | | | |
| Rs-rs | | | | | | |
| TG | 0.433 | 0.17 | 0.52 | 0.68 | 0.481 | 0.499 |
| GC | 0.426 | 0.89 | 0.38 | 0.27 | 0.411 | 0.438 |
| GG | 0.086 | 0.42 | 0.05 | 0.405 | 0.101 | 0.032 |
| TC | 0.055 | 0.038 | 0.05 | 0.405 | 0 | 0.032 |

TABLE 8

PCR and sequencing primers for Human ASMT

| | | | Size | | | |
|---|---|---|---|---|---|---|
| | | | PCR | Ta | | Seq |
| EXON | Size (exon) | Primers | (bp) | (° C.) | comment | primer |
| 1A | 78 | ASMT1AF (SEQ ID NO: 7): | 875 | 67 | 5% DMSO | Fwd and |
| | | GAGCGATTCTTCTGCCTCAGC | | | (1m elong) | rev |
| | | ASMT1AR (SEQ ID NO: 8): | | | | |
| | | TCTGCGCACACTCCCAGGTG | | | | |
| 1B/C | 136 | ASMT1BF (SEQ ID NO: 9): | 820 | 67 | 5% DMSO | Fwd and |
| | coding: 69 | GAGGCAGGAGAATCGCTTGAA | | | (1m elong) | rev |
| | | ASMT1BR (SEQ ID NO: 10): | | | | |
| | | GGCTACATCGTGGGTGTACGTC | | | | |
| 2 | 175 | ASMT2F (SEQ ID NO: 11): | 552 | 58 | | Rev |
| | | TGGTGCAATCTCATTTGACTCTG | | | | |
| | | ASMT2R (SEQ ID NO: 12): | | | | |
| | | GGGTTCATGCCATTCTCCTG | | | | |
| 3 | 130 | ASMT3F1 (SEQ ID NO: 13): | 950 | 64 | 5% DMSO | Fwd and |
| | | CAGCTGTACAAGGCAAGAGGA | | | (1m elong) | rev |
| | | ASMT3R2 (SEQ ID NO: 14): | | | | |
| | | CTTTCACCTCCTCCACTGCCA | | | | |
| 4 | 69 | ASMT4F (SEQ ID NO; 15); | 283 | 55 | | Rev |
| | | GCCTGGGCTACAGAGCTGAAA | | | | |
| | | ASMT4R (SEQ ID NO: 16): | | | | |
| | | CTCCTGGGTTGTGCCATTTG | | | | |
| 5 | 119 | ASMT5F (SEQ ID NO: 17): | 331 | 64 | | Fwd |
| | | CCTGTGGGGTATAGCTCCGTTC | | | | |
| | | ASMT5R (SEQ ID NO; 18): | | | | |
| | | CGCACATGTCAAAGCATCAGA | | | | |
| 6 | 84 | ASMT6F (SEQ ID NO: 19): | 342 | 64 | | Fwd |
| | | AGCTTGCAGTGAGCGGAAATC | | | | |
| | | ASMT6R (SEQ ID NO: 20): | | | | |
| | | GCACCCATCGACTCGTCATTT | | | | |
| 7 | 141 | ASMT7F(SEQ ID NO: 21): | 352 | 64 | | Rev |
| | | TGGGTTGGACCCTTCATGAGT | | | | |
| | | ASMT7R (SEQ ID NO: 22): | | | | |
| | | GTGTTTCCGGGAGTGAGAGGA | | | | |
| 8 | 123 | ASMT8F (SEQ ID NO: 23): | 338 | 64 | | Fwd |
| | | AGCCTGGAAGACCTGGGAAAG | | | | |
| | | ASMT8R (SEQ ID NO: 24): | | | | |
| | | CCTGTGGGATGATTTCAGTGC | | | | |
| 9 | 279 | ASMT9F (SEQ ID NO: 25): | 506 | 64 | | Fwd |
| | coding: 212 | GGTGCCCTGACTGTCCTCTGA | | | | |
| | | ASMT9R (SEQ ID NO: 26): | | | | |
| | | CCATCAGCGTGGTCCTCAGTA | | | | |

All PCRs performed with Qiagen HotstarTaq with the temperature profile: 15 min at 95° 35 cycles of: 30s at 95°. 30s at Ta. 30s-1 min at 72° followed by a final extension step of 10 min at 72°.

TABLE 9
Variations identified in ASD, ADHD and controls

| MTNR1A | | | | MTNR1B | | | |
|---|---|---|---|---|---|---|---|
| Rares | | | | Rares | | | |
| variants | ASD | ADHD | Controls | variants | ASD | ADHD | Co [symbol] |
| Gly166 Glu | 4/79 | 1/87 | 5/100 | Gly24Glu | 8/78 | 8/62 | [symbol] |
| | | | | | 2/78 HO | 1/62 HO | |
| Tyr170X | 0/79 | 1/79 | 0/100 | Arg 231 His | 2/130 | 2/99 | [symbol] |
| Ile189Ile* | 2/79 | 8/87 | 5/100 | Lys 243 Arg | 8/130 | 3/102 | [symbol] |
| | | 2/87 HO | | | | | |
| Ile 212Thr | 2/79 | 0/87 | 0/100 | Arg330 Gln | 1/130 | 0/102 | [symbol] |
| Ala266Val | 2/79 | 4/87 | 4/100 | | | | |
| Arg308Arg | 3/79 | 5/87 | 2/100 | | | | |
| | 1/79 HO | | | | | | |
| Thr315Thr | 5/79 | 5/87 | 2/100 | | | | |
| | 1/79 HO | | | | | | |
| Lys334Asn | 1/79 | 2/87 | 2/100 | | | | |

*P = 0.05 between ADHD and Controls;
HO: homozygotes;
N/A: not available [symbol]
indicates data missing or illegible when filed

### REFERENCES

1. Folstein, S. E. & Rosen-Sheidley, B. Genetics of autism: complex aetiology for a heterogeneous disorder. Nat Rev Genet 2, 943-955 (2001).
2. Veenstra-VanderWeele, J. & Cook, E. H. Molecular genetics of autism spectrum disorder. Mol Psychiatry 9, 819-832 (2004).
3. Bobb, A. J., Castellanos, F. X., Addington, A. M. & Rapoport, J. L. Molecular genetic studies of ADHD: 1991 to 2004. Am J Med Genet B Neuropsychiatr Genet 132, 109-25 (2005).
4. Faraone, S. V. et al. Molecular genetics of attention-deficit/hyperactivity disorder. Biol Psychiatry 57, 1313-23 (2005).
5. American Psychiatric Association. Diagnostic and Statistical Manual of Mental Disorders, 4th Ed. (American Psychiatric Press, Washington D.C., 1994).
6. Biederman, J. & Faraone, S. V. Attention-deficit hyperactivity disorder. Lancet 366, 237-48 (2005).
7. Smalley, S. L. Genetic influences in childhood-onset psychiatric disorders: autism and attention-deficit/hyperactivity disorder. American Journal of Human Genetics 60, 1276-82. (1997).
8. Ozonoff, S. & Jensen, J. Brief report: specific executive function profiles in three neurodevelopmental disorders. J Autism Dev Disord 29, 171-7 (1999).
9. Handen, B. L., Johnson, C. R. & Lubetsky, M. Efficacy of methylphenidate among children with autism and symptoms of attention-deficit hyperactivity disorder. J Autism Dev Disord 30, 245-55 (2000).
10. Smalley, S. L. et al. Genetic linkage of attention-deficit/hyperactivity disorder on chromosome 16p13, in a region implicated in autism. Am J Hum Genet 71, 959-63 (2002).
11. Thomas, N. S. et al. Xp deletions associated with autism in three females. Human Genetics 104, 43-48 (1999).
12. Boycott, K. M. et al. A familial contiguous gene deletion syndrome al Xp22.3 characterized by severe learning disabilities and ADHD. Am J Med Genet A 122, 139-47 (2003).
13. Doherty, M. J. et al. An Xp; Yq translocation causing a novel contiguous gene syndrome in brothers with generalized epilepsy, ichthyosis, and attention deficits. Epilepsia 44, 1529-35 (2003).
14. Yi, H., Donohue, S. J., Klein, D. C. & McBride, O. W. Localization of the hydroxyindole-O-methyltransferase gene to the pseudoautosomal region: implications for mapping of psychiatric disorders. Hum Mol Genet 2, 127-31 (1993).
15. Reiter, R. J. Melatonin: clinical relevance. Best Pract Res Clin Endocrinol Metab 17, 273-85 (2003).
16. Simonneaux, V. & Ribelayga, C. Generation of the melatonin endocrine message in mammals: a review of the complex regulation of melatonin synthesis by norepinephrine, peptides, and other pineal transmitters. Pharmacol Rev 55, 325-95 (2003).
17. Reiter, R. J. Pineal melatonin: cell biology of its synthesis and of its physiological interactions. Endocr Rev 12, 151-80 (1991).
18. Nelson, R. J. & Drazen, D. L. Melatonin mediates seasonal changes in immune function. Ann N Y Acad Sci 917, 404-15 (2000).
19. Nir, I. et al. Brief report: circadian melatonin, thyroid-stimulating hormone, prolactin, and cortisol levels in serum of young adults with autism. J Autism Dev Disord 25, 641-54 (1995).
20. Lord, C. What is melatonin? Is it a useful treatment for sleep problems in autism? J Autism Dev Disord 28, 345-6 (1998).
21. Hayashi, E. Effect of melatonin on sleep-wake rhythm: the sleep diary of an autistic male. Psychiatry Clin Neurosci 54, 383-4 (2000).
22. Kulman, G. et al. Evidence of pineal endocrine hypofunction in autistic children. Neuroendocrinol Lett 21, 31-34 (2000).
23. Pacchierotti, C., Iapichino, S., Bossini, L., Pieraccini, F. & Castrogiovanni, P. Melatonin in psychiatric disorders: a review on the melatonin involvement in psychiatry. Front Neuroendocrinol 22, 18-32 (2001).
24. Yun, A. J., Bazar, K. A. & Lee, P. Y. Pineal attrition, loss of cognitive plasticity, and onset of puberty during the teen years: is it a modern maladaptation exposed by evolutionary displacement? Med Hypotheses 63, 939-50 (2004).
25. Tordjman, S., Anderson, G. M., Pichard, N., Charbuy, H. & Touitou, Y. Nocturnal excretion of 6-sulphatoxymelatonin in children and adolescents with autistic disorder. Biol Psychiatry 57, 134-8 (2005).
26. Ishizaki, A., Sugama, M. & Takeuchi, N. [Usefulness of melatonin for developmental sleep and emotional/behavior disorders—studies of melatonin trial on 50 patients with developmental disorders]. No To Hattatsu 31, 428-37 (1999).
27. Rodriguez, I. R., Mazuruk, K., Schoen, T. J. & Chader, G. J. Structural analysis of the human hydroxyindole-O-methyltransferase gene. Presence of two distinct promoters. J Biol Chem 269, 31969-77 (1994).
28. Chanut E, Nguyen-Legros J, Versaux-Botteri C, Trouvin J H, Launay J M. Determination of melatonin in rat pineal, plasma and retina by high-performance liquid chromatography with electrochemical detection. J Chromatogr B Biomed Sci Appl. 1998 May 8; 709(1):11-8
29. Finocchiaro L M, Nahmod V E, Launay J M. Melatonin biosynthesis and metabolism in peripheral blood mononuclear leucocytes. Biochem J. 1991 Dec. 15; 280 (Pt 3):727-31.

1. A method for determining likelihood of an individual of developing a psychiatric disorder, comprising:
identifying at least one genetic variation in a gene involved in the modulation of melatonin and/or
assaying the mRNA transcript level of said gene and/or,
assaying the enzymatic activity of an enzyme involved in the melatonin biosynthesis pathway in a sample of said individual, and/or
assaying the melatonin level,
whereby identification of a genetic variation and/or decrease in the enzymatic activity and/or a decrease in the transcript level of said gene and/or a decrease in melatonin level as compared to a psychiatric-free individual, is indicative of a likelihood that said individual develops a psychiatric disorder.

2. The method according to claim 1, wherein said gene is ASMT gene.

3. The method according to claim 2, wherein said genetic variation is a point mutation.

4. The method according to claim 3, wherein said point mutation is a mutation at position 17, 81, 210, 306 or 326 as defined by the position in SEQ ID NO:1.

5. The method according to claim 4, wherein said point mutation is a N17K mutation.

6. The method according to claim 4, wherein said point mutation is a K81E mutation.

7. The method according to claim 4, wherein said point mutation is a R210H mutation.

8. The method according to claim 4, wherein said point mutation is a G306A mutation.

9. The method according to claim 4, wherein said point mutation is a L326F mutation.

10. The method according to claim 2, wherein said genetic variation is a splice site mutation.

11. The method according to claim 10, wherein the splice site mutation is IVS5+2T>C.

12. The method according to claim 2, wherein said genetic variation is located in a single nucleotide polymorphism (SNP) of said ASMT gene.

13. The method according to claim 12, wherein said SNP is Rs5989681 or Rs6588809.

14. The method according to claim 3, wherein said point mutation is a mutation at position 17, 81, 228 or 298 as defined by the position in SEQ ID NO:2.

15. The method according to claim 3, wherein said point mutation is a mutation at position 17, 81, 231 or 251 as defined by the position in SEQ ID NO:3.

16. The method according to claim 1, wherein said gene is AA.NAT gene.

17. The method according to claim 16, wherein said genetic variation is a point mutation.

18. The method according to claim 17, wherein said point mutation is a mutation at position 3, 13, 62, 157 or 163 as defined by the position in SEQ ID NO:4.

19. The method according to claim 18, wherein said point mutation is a T3M mutation.

20. The method according to claim 18, wherein said point mutation is a A13S mutation.

21. The method according to claim 18, wherein said point mutation is a V62I mutation.

22. The method according to claim 18, wherein said point mutation is a A157V mutation.

23. The method according to claim 18, wherein said point mutation is a A163V mutation.

24. The method according to claim 1, wherein said enzyme consists of HIOMT (Hydroxyindole-O-methyltransferase) or functional derivatives thereof.

25. The method according to claim 1, wherein said gene is a gene coding for a melatonin receptor.

26-42. (canceled)

43. The method according to claim 25, wherein the melanine receptor consists of MTNR1A or MTNR1B.

44. The method according to claim 25, wherein said genetic variation is a point mutation.

45. The method according to claim 44, wherein said point mutation is a Y170X mutation in the MTNR1A receptor as defined by the position in SEQ ID NO: 5.

46. The method according to claim 1, wherein said psychiatric disorder is selected from the group consisting of autism spectrum disorders (ASD), Attention Deficits and Hyper Activity Disorder (ADHD) and anorexia.

47. A composition for treating and/or preventing a psychiatric disorder in an individual having a defective gene involved in the modulation of melatonin, comprising an acceptable carrier and a therapeutically effective amount of a molecule promoting melatonin modulation.

48. The composition according to claim 47, wherein said molecule is a polypeptide.

49. The composition according to claim 48, wherein said polypeptide is an enzyme.

50. The composition according to claim 49, wherein said enzyme is HIOMT (Hydroxyindole-O-methyltransferase) enzyme or functional derivatives thereof.

51. A method for treating and/or preventing a psychiatric disorder in an individual having a defective gene involved in the modulation of melatonin comprising the administration in said individual of a composition as defined in claim 47.

52. The method according to claim 51, wherein said psychiatric disorder is selected from the group consisting of autism spectrum disorders (ASD), Attention Deficits and Hyper Activity Disorder (ADHD) and anorexia.

53. A method for treating and/or preventing ASD in an individual comprising the administration in said individual of a composition comprising melatonin or a functional derivative thereof.

54. An isolated polynucleotide encoding a polypeptide or functional derivative thereof, wherein said polypeptide or functional derivative thereof comprises a point mutation in the amino acid sequence as defined in SEQ ID NO:1.

55. An isolated polynucleotide encoding a polypeptide or functional derivative thereof, wherein said polypeptide or functional derivative thereof comprises a point mutation in the amino acid sequence as defined in SEQ ID NO:4.

56. An isolated polynucleotide encoding a polypeptide or functional derivative thereof, wherein said polypeptide or functional derivative thereof comprises a point mutation in the amino acid sequence as defined in SEQ ID NO:5.

57. An isolated polynucleotide comprising a regulatory sequence of the ASMT gene, wherein said regulatory sequence comprises an insertion/deletion mutation compared to the wild type regulatory sequence of said ASMT gene.

58. An isolated polynucleotide comprising a regulatory sequence of the AANAT gene, wherein said regulatory sequence comprises an insertion/deletion mutation compared to the wild type regulatory sequence of said AANAT gene.

59. The isolated polynucleotide according to claim 57, wherein said regulatory sequence consists of a promoter.

60. An isolated polypeptide encoded by a polynucleotide as defined in claim 54.

61. The method according to claim 43, wherein said genetic variation is a point mutation.

62. The isolated polynucleotide according to claim 58, wherein said regulatory sequence consists of a promoter.

63. An isolated polypeptide encoded by a polynucleotide as defined in claim 55.

64. An isolated polypeptide encoded by a polynucleotide as defined in claim 56.


SEQUENCE LISTING (36 sequences)

SEQ ID NO: 1 - protein, 373 aa, Homo sapiens
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGTRIKLETIILSKLSQGQKTKHRVFSLIGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 2 - protein, 345 aa, Homo sapiens
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 3 - protein, 298 aa, Homo sapiens
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 4 - protein, 207 aa, Homo sapiens
MSTQSTHPLKPEAPRLPPGIPESPSCQRRHTLPASEFRCLTPEDAVSAFEIEREAFISVLGVCPLYLDEIRHFLTLCPELSLGWFEEGCLVAFIIGSLWDKERLMQESLTLHRSGGHIAHLHVLAVHRAFRQQGRGPILLWRYLHHLGSQPAVRRAALMCEDALVPFYERFSFHAVGPCAITVGSLTFMELHCSLRGHPFLRRNSGC

SEQ ID NO: 5 - protein, 350 aa, Homo sapiens [sequence omitted]

SEQ ID NO: 6 - protein, 362 aa, Homo sapiens
MSENGSFANCCEAGGWAVRPGWSGAGSARPSRTPRPPWVAPALSAVLIVTTAVDVVGNLLVILSVLRNRKLRNAGNLFLVSLALADLVVAFYPYPLILVAIFYDGWALGEEHCKASAFVMGLSVIGSVFNITAIAINRYCYICHSMAYHRIYRRWHTPLHICLIWLLTVVALLPNFFVGSLEYDPRIYSCTFIQTASTQYTAAVVVIHFLLPIAVVSFCYLRIWVLVLQARRKAKPESRLCLKPSDLRSFLTMFVVFVIFAICWAPLNCIGLAVAINPQEMAPQIPEGLFVTSYLLAYFNSCLNAIVYGLLNQNFRREYKRILLALWNPRHCIQDASKGSHAEGLQSPAPPIIGVQHQADAL

SEQ ID NO: 7 - DNA, 21 bp, Artificial Sequence
  Description of Artificial Sequence Synthetic primer
gagcgattcttctgcctcagc

SEQ ID NO: 8 - DNA, 20 bp, Artificial Sequence
  Description of Artificial Sequence Synthetic primer
tctgcgcacactcccaggtg

SEQ ID NO: 9 - DNA, 21 bp, Artificial Sequence
  Description of Artificial Sequence Synthetic primer
gaggcaggagaatcgcttgaa

SEQ ID NO: 10 - DNA, 22 bp, Artificial Sequence
  Description of Artificial Sequence Synthetic primer
ggctacatcgtgggtgtacgtc

SEQ ID NO: 11 - DNA, 23 bp, Artificial Sequence
  Description of Artificial Sequence Synthetic primer
tggtgcaatctcatttgactctg

SEQ ID NO: 12 - DNA, 20 bp, Artificial Sequence
  Description of Artificial Sequence Synthetic primer
gggttcatgccattctcctg

SEQ ID NO: 13 - DNA, 21 bp, Artificial Sequence
  Description of Artificial Sequence Synthetic primer
cagctgtacaaggcaagagga

SEQ ID NO: 14 - DNA, 21 bp, Artificial Sequence
  Description of Artificial Sequence Synthetic primer
ctttcacctcctccactgcca

SEQ ID NO: 15 - DNA, 21 bp, Artificial Sequence
  Description of Artificial Sequence Synthetic primer
gcctgggctacagagctgaaa

SEQ ID NO: 16 - DNA, 20 bp, Artificial Sequence
  Description of Artificial Sequence Synthetic primer
ctcctgggttgtgccatttg

SEQ ID NO: 17 - DNA, 22 bp, Artificial Sequence
  Description of Artificial Sequence Synthetic primer
cctgtggggtatagctccgttc

SEQ ID NO: 18 - DNA, 21 bp, Artificial Sequence
  Description of Artificial Sequence Synthetic primer
cgcacatgtcaaagcatcaga

SEQ ID NO: 19 - DNA, 21 bp, Artificial Sequence
  Description of Artificial Sequence Synthetic primer
agcttgcagtgagcggaaatc

SEQ ID NO: 20 - DNA, 21 bp, Artificial Sequence
  Description of Artificial Sequence Synthetic primer
gcacccatcgactcgtcattt

SEQ ID NO: 21 - DNA, 21 bp, Artificial Sequence
  Description of Artificial Sequence Synthetic primer
tgggttggacccttcatgagt

SEQ ID NO: 22 - DNA, 21 bp, Artificial Sequence
  Description of Artificial Sequence Synthetic primer
gtgtttccgggagtgagagga

SEQ ID NO: 23 - DNA, 21 bp, Artificial Sequence
  Description of Artificial Sequence Synthetic primer
agcctggaagacctgggaaag

SEQ ID NO: 24 - DNA, 21 bp, Artificial Sequence
  Description of Artificial Sequence Synthetic primer
cctgtgggatgatttcagtgc

SEQ ID NO: 25 - DNA, 21 bp, Artificial Sequence
  Description of Artificial Sequence Synthetic primer
ggtgccctgactgtcctctga

SEQ ID NO: 26 - DNA, 21 bp, Artificial Sequence
  Description of Artificial Sequence Synthetic primer
ccatcagcgtggtcctcagta

SEQ ID NO: 27 - DNA, 430 bp, Homo sapiens
  CDS (359)..(427)
caggacctgctcaatccataagacgattaatgagtgctaatgatgggtgagcatattggtttgggatcagttctacagctgatgagtcaagggctcgggggggcctgatgcagactattttagggctgcggaggggcgcccagaaagccagggaatcgccacacgctttgtgatcccctaatacggttaatgcaaatttctagagtgatccgtctttgtttgagtactgggcaggcagcagggagagtcaggcagcagctgtgagcgggtggctcttccccaccttgccagcaggctctgtgctccttgaagcaagcgctccagaggctccggaagccacggctggattggagacaagatgggatcctcagaggaccaggcctatcgcctccttaatgactacgccaacggcttcatggtgtcccaggta

SEQ ID NO: 28 - protein, 23 aa, Homo sapiens
MGSSEDQAYRLLNDYANGFMVSQ

SEQ ID NO: 29 - DNA, 59 bp, Homo sapiens
ttcccacttatgtgtgaccttggtggyaagtacccctcaccactaatacctcggaggtg

SEQ ID NO: 30 - protein, 65 aa, Homo sapiens
WADGKCSHLLERIYHTCKPGGAILVIESLLDEDRRGPLLTQFYSLNMLVQTEGQERTPTHYHMLL

SEQ ID NO: 31 - protein, 65 aa, Macaca sp.
WADGKCSHLLERVYHTCKPGGGILVIESLLDEDRRGPLLTQLYSLNMLVQTEGQERTPTHYHMLL

SEQ ID NO: 32 - protein, 65 aa, Bovine sp.
WTDAKCSHLLQRVYRACRTGGGILVIESLLDTDGRGPLTTLLYSLNMLVQTEGRERTPAEYRALL

SEQ ID NO: 33 - protein, 65 aa, Rattus sp.
WADGACVELLGRLHRACTSSGALLLVEAVLAKGGAGPLRSLLLSLNMMLQAEGWERQASDYRNLA

SEQ ID NO: 34 - protein, 65 aa, Gallus sp.
WDDKKCRQLLAEVYKACRPGGGVLLVESLLSEDRSGPVETQLYSLNMLVQTEGKERTAVEYSELL

SEQ ID NO: 35 - protein, 62 aa, Danio sp.
WSDEKLHVLLSKLCKMWTPGCGLLVSEILLDEERRRPSRALLQALSMTEGKQRSTAEYSALL

SEQ ID NO: 36 - protein, 65 aa, Fugu sp.
WTDQRCLELLRRVHGACRPGGSVLLVEALLYEDGSGPLTAQLYSLNMLVQTEGRERSAAQYAALL